(19) United States
(12) Patent Application Publication
(10) Pub. No.: US 2002/0039723 A1
(43) Pub. Date: Apr. 4, 2002

(54) BIOCATALYTIC METHODS FOR SYNTHESIZING AND IDENTIFYING BIOLOGICALLY ACTIVE COMPOUNDS

(76) Inventors: J. WESLEY FOX, SCHAUMBURG, IL (US); JONATHAN S. DORDICK, IOWA CITY, IA (US); DOUGLAS S. CLARK, OAKLAND, CA (US)

Correspondence Address: MICHEAL L. GOLDMAN, NIXON PEABODY LLP, CLINTON SQUARE , P.O. BOX 31051, ROCHESTER, NY, 14603, US

(21) Appl. No.: 09/246,267

(22) Filed: Feb. 8, 1999

Related U.S. Application Data
(63) Continuation of application No. 08/383,715, filed on Feb. 3, 1995.
Application No. 08/383,715 is a continuation of application No. 08/106,279, filed on Aug. 13, 1993, now abandoned.

(51) Int. Cl. C12Q 1/00; G01N 33/567; G01N 33/543

(52) U.S. Cl. 435/4; 435/41; 436/501; 436/506; 436/518; 422/50; 422/63; 422/68.1

(57) ABSTRACT

This invention encompasses methods for producing a library of modified starting compounds by use of biocatalytic reactions on a starting compound and identifying the modified starting compound with the optimum desired activity. The method is useful in producing modified pharmaceutical compounds with desired specific activity.

### BACKGROUND OF THE INVENTION

(a) Field of the Invention

This invention is in the field of synthesizing and identifying biologically active compounds.

(b) Description of the Prior Art

The prior art is repleat with examples of chemically or microbially synthesizing compounds with biological activity. The goal of these efforts is the discovery of new and improved pharmaceutical compounds.

The discovery of new pharmaceutical compounds is for the most part a trial and error process. So many diverse factors constitute an effective pharmaceutical compound that- it is extremely difficult to reduce the discovery process to a systematic approach. Typically, thousands of organic compounds must be isolated from biological sources or chemically synthesized and tested before a pharmaceutical compound is found.

Synthesizing and testing new compounds for biological activity, which is the first step in identifying a new synthetic drug, is a time consuming and expensive undertaking. Typically, compounds must by synthesized, purified, tested and quantitatively compared to other compounds in order to identify active compounds or identify compounds with optimal activity. The synthesis of new compounds is accomplished for the most part using standard chemical methods. Such methods provide for the synthesis of virtually any type of organic compound; however, because chemical reactions are non-specific, these syntheses require numerous steps and multiple purifications before a final compound is produced and ready for testing.

New biological and chemical approaches have recently been developed which provide for the synthesis and screening of large libraries of small peptides and oligonucleotides. These methods provide for the synthesis of a broad range of chemical compounds and provide the means to potentially identify biologically active compounds. The chemistries for synthesizing such large numbers of these natural and non-naturally occurring polymeric compounds is complicated, but manageable because each compound is synthesized with the same set of chemical protocols, the difference being the random order in which amino acids or nucleotides are introduced into the reaction sequence.

Fodor, S. P. A. et al (1990) Science 251, 767-773, describe methods for discovering new peptide ligands that bind to biological receptors. The process combines solid-phase chemistry and photolithography to achieve a diverse array of small peptides. This work and related works are also described in Fodor WO Patent #9,210,092, Dower WO #9,119,818, Barrett WO #9,107,087 and Pirrung WO#9,015,070.

Houghten, R. A. et al. (1991) Nature 354, 84-86, describe an approach that synthesizes libraries of free peptides along with an iterative selection process that permits the systematic identification of optimal peptide ligands. This work is also described in Appel WO Patent #9,209,300.

Lam, K. S., et al. (1991) Nature 354, 82-84, describe a method that provides for the systematic synthesis and screening of peptide libraries on a solid-phase microparticle support on the basis of a ‘one-bead, one-peptide’ approach.

Cwirla, S. E., et al (1990) Proc. Natl. Acad. Sci USA 87, 6378-6382, describe a method for constructing a library of peptides on the surface of a phage by cloning randomly synthesized oligonucleotides into the 5′ region of specific phage genes resulting in millions of different hexapeptides expressed at the N terminus of surface proteins.

These methods accelerate the identification of biologically active peptides and oligonucleotides. However, peptides and oligonucleotides have poor bioavailability and limited stability in vivo, which limits their use as therapeutic agents. In general, non-biological compounds which mimic the structure of the active peptides and oligonucleotides must be synthesized based on the approximated three dimensional structure of the peptide or oligonucleotide and tested before an effective drug structure can be identified.

Bunin et al., J. Am. Chem. Soc. (1992) 114, 10997-10998 describe the synthesis of numerous 1,4 benzodiazapine derivatives using solid phase synthesis techniques.

### SUMMARY OF THE INVENTION

The present invention is used to synthesize a library of non-biological organic compounds from a starting compound and identify individual compounds within the library which exhibit biological activity. Unlike peptides and oligonucleotides, non-biological organic compounds comprise the bulk of proven therapeutic agents. The invention can be used to directly identify new drug candidates or optimize an established drug compound which has sub-optimal activity or problematic side effects. This is accomplished through the use of highly specific biocatalytic reactions.

Enzymes are highly selective catalysts. Their hallmark is the ability to catalyze reactions with exquisite stereo-, regio-, and chemo-selectivities that are unparalleled in conventional synthetic chemistry. Moreover, enzymes are remarkably versatile. They can be tailored to function in organic solvents, operate at extreme pH's and temperatures, and catalyze reactions with compounds that are structurally unrelated to their natural, physiological substrates.

The reactivity of enzymes can be highly specific to an individual structural moiety found on a particular compound or in some cases to a structural group present in a wide range of compounds, albeit in a selective manner with respect to chirality, position, or chemistry. Enzymes are also capable of catalyzing many diverse reactions unrelated to their physiological function in nature. For example, peroxidases catalyze the oxidation of phenols by hydrogen peroxide. Peroxidases can also catalyze hydroxylation reactions that are not related to the native function of the enzyme. Other examples are proteases which catalyze the breakdown of polypeptides. In organic solution some proteases can also acylate sugars, a function unrelated to the native function of these enzymes.

The present invention exploits the unique catalytic properties of enzymes. Whereas the use of biocatalysts (i.e., purified or crude enzymes, non-living or living cells) in chemical transformations normally requires the identification of a particular biocatalyst that reacts with a specific starting compound, the present invention uses selected biocatalysts and reaction conditions that are specific for functional groups that are present in many starting compounds. Each biocatalyst is specific for one functional group, or several related functional groups, and can react with many starting compounds containing this functional group.

The biocatalytic reactions produce a population of derivatives from a single starting compound. These derivatives can be subjected to another round of biocatalytic reactions to produce a second population of derivative compounds. Thousands of variations of the original compound can be produced with each iteration of biocatalytic derivatization.

Enzymes react at specific sites of a starting compound without affecting the rest of the molecule, a process which is very difficult to achieve using traditional chemical methods. This high degree of biocatalytic specificity provides the means to identify a single active compound within the library. The library is characterized by the series of biocatalytic reactions used to produce it, a so called “biosynthetic history”. Screening the library for biological activities and tracing the biosynthetic history identifies the specific reaction sequence producing the active compound. The reaction sequence is repeated and the structure of the synthesized compound determined. This mode of identification, unlike other synthesis and screening approaches, does not require immobilization technologies, and compounds can be tested free in solution using virtually any type of screening assay. It is important to note, that the high degree of specificity of enzyme reactions on functional groups allows for the “tracking” of specific enzymatic reactions that make up the biocatalytically produced library.

Many of the procedural steps are performed using robotic automation enabling the execution of many thousands of biocatalytic reactions and screening assays per day as well as ensuring a high level of accuracy and reproducibility. As a result, a library of derivative compounds can be produced in a matter of weeks which would take years to produce using current chemical methods.

The present invention specifically incorporates a number of diverse technologies such as: (1) the use of enzymatic reactions to produce a library of drug candidates; (2) the use of enzymes free in solution or immobilized on the surface of particles; (3) the use of receptors (hereinafter this term is used to indicate true receptors, enzymes, antibodies and other biomolecules which exhibit affinity toward biological compounds, and other binding molecules to identify a promising drug candidate within a library); (4) the automation of all biocatalytic processes and many of the procedural steps used to test the libraries for desired activities, and (5) the coupling of biocatalytic reactions with drug screening devices which can immediately measure the binding of synthesized compounds to localized or immobilized receptor molecules and thereby immediately identify specific reaction sequences giving rise to biologically active compounds.

Specifically, the present invention encompasses a method for drug identification comprising:

(a) conducting a series of biocatalytic reactions by mixing biocatalysts with a starting compound to produce a reaction mixture and thereafter a library of modified starting compounds;

(b) testing the library of modified starting compounds to determine if a modified starting compound is present within the library which exhibits a desired activity;

(c) identifying the specific biocatalytic reactions which produce the modified starting compound of desired activity by systematically eliminating each of the biocatalytic reactions used to produce a portion of the library and testing the compounds produced in the portion of the library for the presence or absence of the modified starting compound with the desired activity; and

(d) repeating the specific biocatalytic reactions which produce the modified compound of desired activity and determining the chemical composition of the reaction product.

More specifically, the enzymatic reactions are conducted with a group of enzymes that react with distinct structural moieties found within the structure of a starting compound. Each enzyme is specific for one structural moiety or a group of related structural moieties. Furthermore, each enzyme reacts with many different starting compounds which contain the distinct structural moiety.

### BRIEF DESCRIPTION OF THE DRAWINGS

FIG. 1 shows the starting active compound AZT with four potential sites for biocatalytic derivatization and eight possible biocatalytic reactions that can be used to produce a library of derivative compounds.

FIG. 2 shows an automated system employing robotic automation to perform hundreds of biocatalytic reactions and screening assays per day.

FIG. 3 illustrates the tracking of biocatalytic reactions to identify the sequence of reactions producing an active compound, which can subsequently be used to produce and identify the structure of the active compound.

FIG. 4a illustrates biocatalytic modification of castanospermine.

FIG. 4b illustrates biocatalytic modifications to methotrexate.

### DETAILED DESCRIPTION OF THE INVENTION

While the invention will be described in connection with certain preferred embodiments, it will be understood that the description does not limit the invention to these particular embodiments. In fact, it is to be understood that all alternatives, modifications and equivalents are included and are protected, consistent with the spirit and scope of the inventions as defined in the appended claims.

The preferred embodiments of the invention are set forth in the following example:

a) A starting compound such as AZT is chosen which exhibits drug activity or is believed to exhibit drug activity for a given disease or disorder. The compound is analyzed with respect to its functional group content and its potential for structural modifications using selected biocatalytic reactions. Functional groups which can be chemically modified using the selected biocatalytic reactions are listed in Table I. One of more of these functional groups are present in virtually all organic compounds. A partial list of possible enzymatic reactions that can be used to modify these functional groups is presented in Table II.

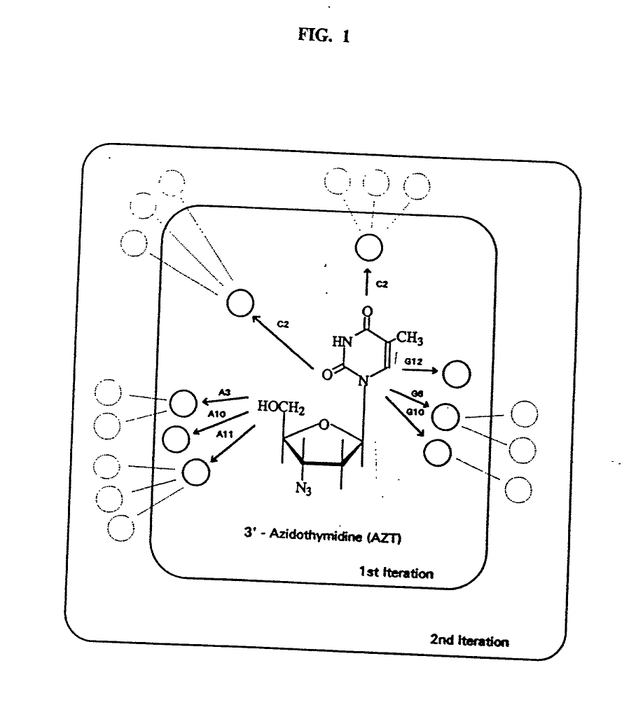

b) A strategy is developed to systematically modify these functional groups using selected biocatalytic reactions and produce a library of derivative compounds to be screened for biological activity. AZT contains four functional groups which are selected for biocatalytic modification: a primary hydroxyl, two carbonyls and a tertiary amine. The biosynthetic strategy is designated in the form of biocatalytic “reaction box” numbers which correspond to specific types of biocatalytic reactions acting on specific functional groups present in the starting compound. These “reaction boxes” are listed in Table III. The following biocatalytic “reaction boxes” are selected to synthesize an AZT derivative library: A3, A10, A11, C2, G6, G10 and G12. FIG. 1 illustrates the reaction of AZT with these selected biocatalytic reaction boxes.

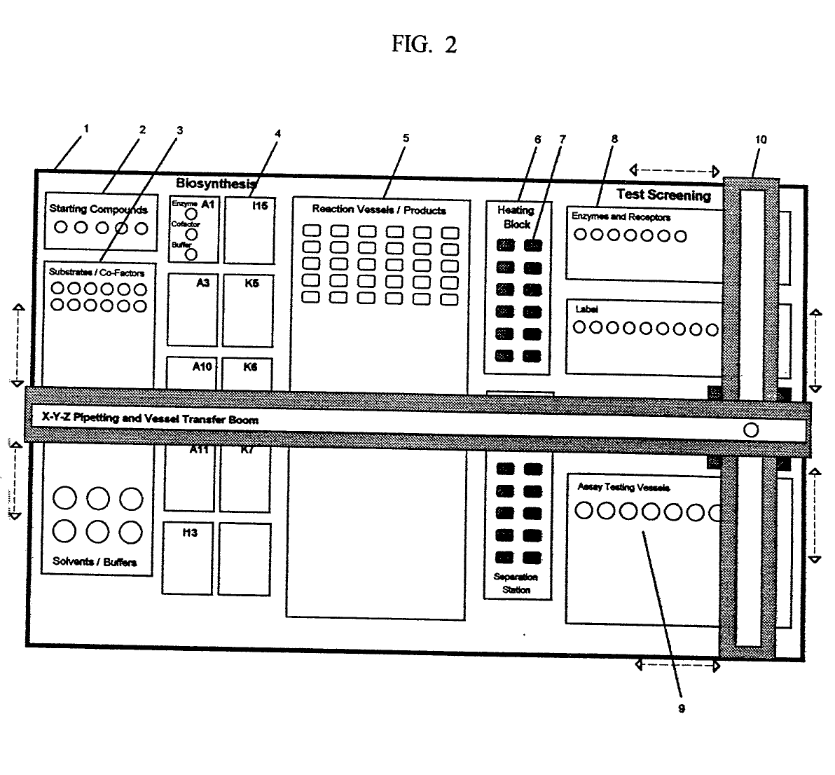

c) The biocatalytic reaction boxes are entered into an automated system which is shown in FIG. 2. The system is programmed to automatically execute the aforementioned biocatalytic reactions and synthesize a library of derivative products. A single automated system in capable of performing hundreds of pre-programmed biocatalytic reactions per day. We can estimate the total number of compounds that can be produced by analyzing the reaction products produced in each “reaction box” and multiplying the results. Table IV details the number of potential reaction products produced in each reaction box and the resulting total number of possible compounds produced. In the case of AZT, up to 1.75×10¹¹ new compounds can be synthesized. It should be pointed out that this compares very favorably to peptide libraries. For example, a library of hexapeptides will contain 20⁶ or 64 million compounds. This is a mere fraction, about 0.04% of the compounds that are possible using the biosynthetic approach described herein. Table V lists the results of a similar analysis on eleven other starting drug compounds. As shown in this table, the biocatalytic reactions can generate huge numbers of derivative compounds for drug screening.

d) The synthesized library of new compounds is assayed using enzyme inhibition assays, receptor- binding assays, immunoassays, and/or cellular assays to identify biologically active compounds. Before assaying the library of derivative compounds, any remaining AZT present in the library is either removed or inhibited to simplify the interpretation of screening assay results. This is easily accomplished by HPLC or the addition of a monoclonal antibody specific for the starting compound. Numerous in vitro assays are available that test for anti-viral, anti-cancer, anti-hypertensive and other well known pharmacological activities. Some of these assays are listed is Table VI. Most of these assays are also performed on the automated system.

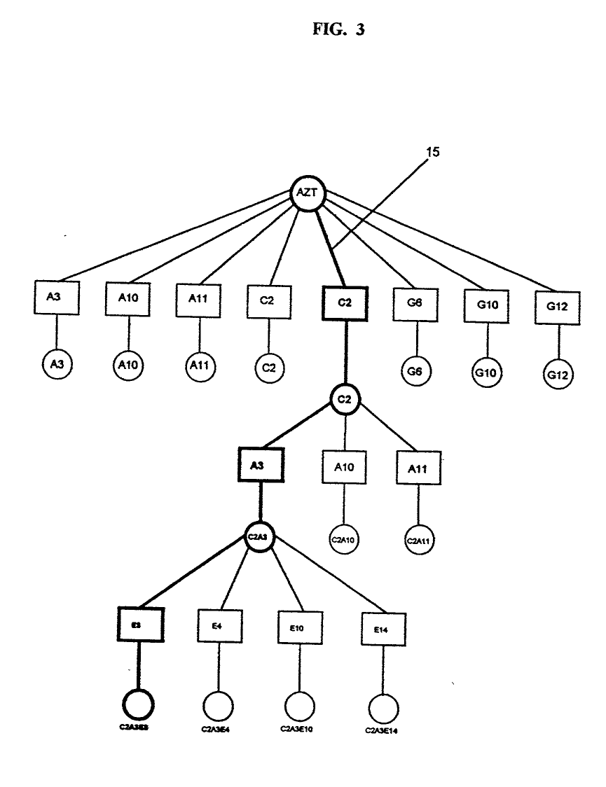

e) Libraries which test positive are further analyzed using a biocatalytic tracking protocol which quickly identifies the specific sequence of reactions responsible for the synthesis of the compound testing positive in the screening assay. The high degree of specificity exhibited by biocatalysts enables this approach to be easily performed. The library is characterized by the series of biocatalytic reactions used to produce it, a so called “biosynthetic history”. Portions of the library are screened for biological activity until the specific reaction sequence producing the active compound is identified. FIG. 3 illustrates this tracking process. For example, the dark line path 15 illustrates the reaction pathway to the most active compound. The reaction sequence is repeated to produce a sufficient amount of product for chemical analysis. The specificity of the biocatalytic reactions also permits the accurate duplication of the reaction pathway producing the active compounds. The structure of the active compound is qualitatively determined by analyzing the starting compounds, substrates and identified biocatalytic reaction sequence. The structure is then confirmed using gas chromatography, mass spectroscopy, NMR spectroscopy and other organic analytical methods. This mode of identification eliminates the need for product purification and also reduces the amount of test screening required to identify a promising new drug compound. This process dramatically reduces the time necessary to synthesize and identify new drug compounds. In addition, this mode of active compound identification does not require immobilization technologies, and compounds can be tested free in solution under in vivo like conditions using virtually any type of screening assay (receptor, enzyme inhibition, immunoassay, cellular, animal model).

Those skilled in the pharmaceutical arts will recognize the large number of biocatalytic conversions such as those listed in Table II and Table III, as well as the in vitro drug screening assays listed in Table VI.

Those skilled in the pharmaceutical arts will recognize that biocatalytic reactions are optimized by controlling or adjusting such factors as solvent, buffer, pH, ionic strength, reagent concentration and temperature.

The biocatalysts used in the biocatalytic reactions may be crude or purified enzymes, cellular lysate preparations, partially purified lysate preparations, living cells or intact non-living cells, used in solution, in suspension, or immobilized on magnetic or non-magnetic surfaces.

In addition, non-specific chemical reactions may also be used in conjunction with the biocatalytic reaction to obtain the library of modified starting compounds. Examples of such non-specific chemical reactions include: hydroxylation of aromatics; oxidation reactions; reduction reactions; hydration reactions; dehydration reactions; hydrolysis reactions; acid/based catalyzed esterification; transesterification; aldol condensation; reductive amination; ammonolysis; dehydrohalogenation; halogenation; acylation; acyl substitution; aromatic substitution; Grignard synthesis; Friedel-Crafts acylation.

The biocatalytic reaction can be performed with a biocatalyst immobilized to magnetic particles forming a magnetic biocatalyst. The method of this embodiment is performed by initiating the biocatalytic reaction by combining the immobilized biocatalyst with substrate(s), cofactors(s) and solvent/buffer conditions used for a specific biocatalytic reaction. The magnetic biocatalyst is removed from the biocatalytic reaction mixture to terminate the biocatalytic reaction. This is accomplished by applying an external magnetic field causing the magnetic particles with the immobilized biocatalyst to be attracted to and concentrate at the source of the magnetic field, thus effectively separating the magnetic biocatalyst from the bulk of the biocatalyst reaction mixture. This allows for the transferral of the reaction mixture minus the magnetic biocatalyst from a first reaction vessel to a second reaction vessel, leaving the magnetic biocatalyst in the first reaction vessel. A second biocatalytic reaction is conducted completely independent-of the first biocatalytic reaction, by adding a second biocatalyst immobilized to magnetic particles to the second reaction vessel containing the biocatalytic reaction mixture S transferred from the first reaction vessel. Finally, these steps are repeated to accomplish a sequential series of distinct and independent biocatalytic reactions, producing a corresponding series of modified starting compounds.

The biocatalytic reactions can also be performed using biocatalysts immobilized on any surface which provides for the convenient addition and removal of biocatalyst from the biocatalytic reaction mixture thus accomplishing a sequential series of distinct and independent biocatalytic reactions producing a series of modified starting compounds.

The biocatalytic reactions can also be used to derivatize known drug compounds producing new derivatives of the drug compound and select individual compounds within this library that exhibit optimal activity. This is accomplished by the integration of a high affinity receptor into the biocatalytic reaction mixture, which is possible because of the compatibility of the reaction conditions used in biosynthesis and screening. The high affinity receptor is added to the reaction mixture at approximately one half the molar concentration of the starting active compound, resulting in essentially all of the receptor being bound with the starting active compound and an equal molar concentration of starting active compound free in solution and available for biocatalytic modification. If the biocatalytic reaction mixture produces a derivative which possesses a higher binding affinity for the receptor, which can translate into improved pharmacological performance, this derivative will displace the bound starting active compound and remain complexed with the receptor, and thus be protected from further biocatalytic conversions. At the end of the experiment, the receptor complex is isolated, dissociated and the bound compound analyzed. This approach accomplishes the identification of an improved version of the drug compound without the need to purify and test each compound individually.

The biocatalytic reactions and in vitro screening assays can be performed with the use of an automated robotic device. The automated robotic device having:

(a) an XY table with an attached XYZ pipetting boom to add volumetric amounts of enzyme, substrate, cofactor, solvent solutions and assay reagents from reagent vessels positioned on the XY table to reaction and assay vessels positioned on the same XY table;

(b) an XYZ reaction-vessel transfer boom attached to the same XY table used to transfer reaction and assay vessels positioned on the XY table to different locations on the XY table;

(c) a temperature incubation block attached to the same XY table to house the reaction and assay vessels during reaction incubations and control the temperature of the reaction mixtures;

(d) a magnetic separation block attached to the same XY table to separate the biocatalyst immobilized to magnetic particles from the biocatalytic mixture by applying an external magnetic field causing the magnetic particles to be attracted to and concentrate at the source of the magnetic filed, thus effectively separating them from the bulk of the biocatalytic reaction mixture; and

(e) a programmable microprocessor interfaced to the XYZ pipetting boom, and XYZ reaction-vessel transfer boom, the temperature block and the magnetic separation block to precisely control and regulate all movements and operations of these functional units in performing biocatalytic reactions to produce modified starting compounds and assays to determine desired activities.

FIG. 2 illustrates the automated robotic device of this invention. Mounted in the frame 1 of the system are containers for starting compounds 2, and containers for reagents 3 such as enzymes, cofactors, and buffers. There are specific biosynthesis boxes 4 which contain reagents for various classes of reactions. The frame also has arrays of reaction vessels 5, and a heating block 6 with wells 7 for conducting reactions at a specific temperature. The frame has an area 8 for reagents for screening test 8 which contains reagents used for conducting screening tests, and area 9 which contains assay vessls for conducting screening tests, the automated system uses a X-Y-Z pipetting and vessel transfer boom 10 to dispense all reagents and solutions, and transfer reaction vessels.

In operation the X-Y-Z reaction-vessels transfer boom can deliver starting compounds and reagents to specific locations for making specific modified starting compounds which in turn can be delivered to specific locations for conducting assays. In this way the process of making modified starting compounds and testing for optimum activity is largely automated.

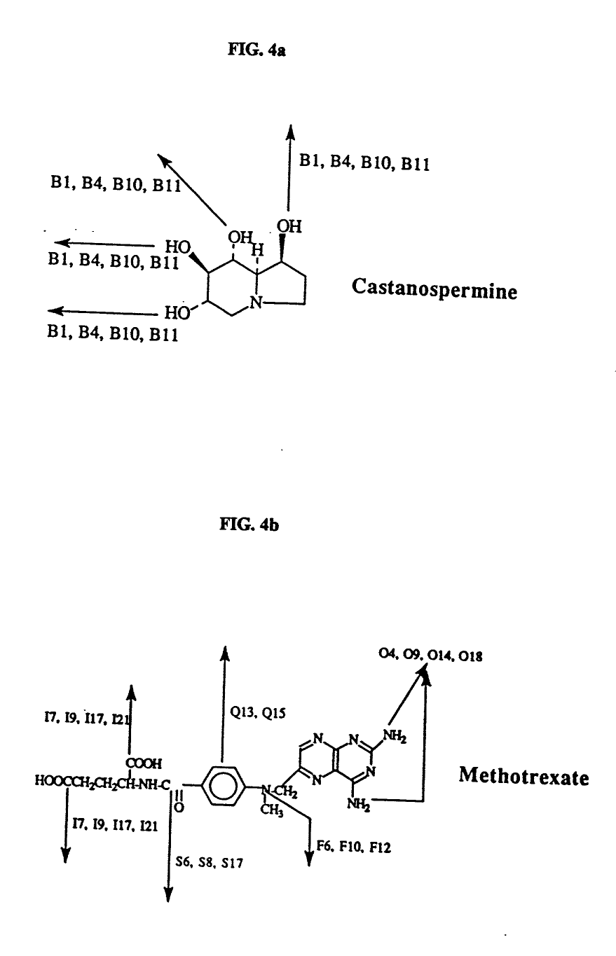

FIGS. 4a and 4b illustrate derivatization of castanospermine and methotrexate. All of these embodiments utilize the biocatalytic conversions set out in Table II and the assays set out in Table VI.

While the invention as described herein is directed to the development of drugs, those skilled in the biological arts will recognize that the methods of this invention are equally applicable to other biologically active compounds such as food additives, pesticides, herbicides, and plant and animal growth hormones.

TABLE I
Major Functional Groups Available for Biocatalytic Modification*

| | |
|---|---|
| A. | Hydroxyl Groups -- These groups can undergo numerous reactions |
| | including oxidation to aldehydes or ketones (1.1), acylation |
| | with ester donors (2.3, 3.1), glycosidic bond formation (2.4, |
| | 3.2, 5.3), and etherification (2.1, 3.3). Potential for stereo- |
| | and regio-selective synthesis as well as prochiral specificity. |
| B. | Aldehydes and Ketones -- These groups can undergo selective |
| | reduction to alcohols (1.1). This may then be followed by |
| | modifications of hydroxyl groups. |
| C. | Amino Groups -- These groups can undergo oxidative deamination |
| | (1.4), N-dealkylation (1.5, 1.11), transfered to other compounds |
| | (2.6), peptide bond synthesis (3.4, 6,3), and acylation with ester |
| | donors (2.3, 3.1). |
| D. | Carboxyl Groups -- These groups can be decarboxylated (1.2, 1.5, |
| | 4.1), and esterified (3.1, 3.6). |
| E. | Thiol Groups -- These groups can undergo reactions similar to |
| | hydroxyls, such as thioester formation (2.8, 3.1), thiol |
| | oxidation (1.8), and disulfide formation (1.8). |
| F. | Aromatic Groups -- These groups can be hydroxylated (1.11, 1.13, |
| | 1.14), and oxidatively cleaved to diacids (1.14). |
| G. | Carbohydrate Groups -- These groups can be transfered to hydroxyls |
| | and phenols (2.4, 3.2, 5.3), and to other carbohydrates (2.4). |
| H. | Ester and Peptide Groups -- These groups can be hydrolyzed (3.1, |
| | 3.4, 3.5, 3.6, 3.9), and transesterified (or interesterified) |
| | (3.1, 3.4). |
| I. | Sulfate and Phosphate Groups -- These groups can be hydrolyzed |
| | (3.1, 3.9), transfered to other compounds (2.7, 2.8), and esterified |
| | (3.1). |
| J. | Halogens -- These groups can be oxidatively or hydrolytically |
| | removed (1.11, 3.8), and added (1.11). |
| K. | Aromatic Amines and Phenols -- These groups can be acylated (2.3, |
| | 3.1) or oxidatively polymerized (1.10, 1.1 1, 1.14). |

*Numbers in parentheses correspond to the EC (Enzyme Commission) categorization of enzymes and enzyme classes.

TABLE II
Biocatalytic Reactions Used to Modify Functional Groups

| | |
|---|---|
| 1. | Oxidation of primary and secondary alcohols; Reduction of |
| | aldehydes and ketones. |
| | Reaction Boxes: A1, B1, C2, D2 |
| | Enzyme Class: 1.1. Dehydrogenases, Dehydtratases, Oxidases |
| | Representative Enzymes: |
| | Alcohol Dehydrogenase |
| | Glycerol Dehydrogenase |
| | Chycerol-3-Phosphate Dehydrogenase |
| | Xylulose Reductase |
| | Polyol Dehydrogenase |
| | Sorbitol Dehydrogenase |
| | Glyoxylate Reductase |
| | Lactate Dehydrogenase |
| | Glycerate Dehydrogenase |
| | β-Hydroxybutyrate Dehydrogenase |
| | Malate Dehydrogenase |
| | Glucose Dehydrogenase |
| | Glucose-6-Phosphate Dehydrogenase |
| | 3a- and 3B-Hydroxysteroid Dehydrogenase |
| | 3a-, 20B-Hydroxsteroid Dehydrogenase |
| | Fucose Dehydrogenase |
| | Cytochrome-Dependent Lactate Dehydrogenase |
| | Galactose Oxidase |
| | Glucose Oxidase |
| | Cholesterol Oxidase |
| | Alcohol Oxidase |
| | Glycolate Oxidase |
| | Xanthine Oxidase |
| | Fructose Dehydrogenase |
| | Cosubstrates/Cofactors: NAD(P)(H) |
| 2. | Acylation of primary and secondary alcohols. |
| | Reaction Boxes: A3, B4 |
| | Enzyme Classes: 3.1, 3.4, 3.5, 3.6 |
| | Representative Enzymes: Esterases, lipases, proteases, sulfatases, |
| | phosphatases, acylases, lactamases, mucleases, acyl transferases |
| | Esterases |
| | Lipases |
| | Phospholipase A |
| | Acetylesterase |
| | Acetyl Cholinesterase |
| | Butyryl Cholinesterase |
| | Pectinesterase |
| | Cholesterol Esterase |
| | Glyoxalase II |
| | Alkaline Phosphatase |
| | Acid Phosphatase |
| | A Variety of nucleases |
| | Glucose-6-Phosphatase |
| | Fructose 1,6-Diphosphatase |
| | Ribonuclease |
| | Deoxyribonuclease |
| | Sulfatase |
| | Chondro-4-Sulfarase |
| | Chondro-6-Sulfarase |
| | Leucine Aminopeptidase |
| | Carboxypeptidase A |
| | Carboxypeptidase B |
| | Carboxypeptidase Y |
| | Carboxypeptidase W |
| | Prolidase |
| | Cathepsin C |
| | Chymotrypsin |
| | Trysin |
| | Elastase |
| | Subtilisin |
| | Papain |
| | Pepsin |
| | Ficin |
| | Bromclaim |
| | Rennin |
| | Proteinase A |
| | Collagenase |
| | Urokinase |
| | Asparaginase |
| | Glutaminase |
| | Urease |
| | Acylase I |
| | Penicillinase |
| | Cephalosporinase |
| | Creatininase |
| | Guanase |
| | Adenosine Deaminase |
| | Creamine Deaminase |
| | R-O-CO-R′ |
| | Where R = alkyl, vinyl, isopropenyl, haloalkyl, aryl, |
| | derivatives of aryl (i.e., nitrophenyl) and R′ can be |
| | any alkyl or aryl group with or without derivatives. Such |
| | derivatives include halogens, charged functional groups (i.e., |
| | acids, sulfates, phosphates, amines, etc.), glycols (protected |
| | or unprotected), etc. |
| 3. | Transglycosylation of primary and secondary alcohols. |
| | Reaction Boxes: A10, B10 |
| | Enzyme Class: 2.4, 3.2 |
| | Representative Enzymes: |
| | Phosphorylase a |
| | Phosphorylase b |
| | Dextransucrase |
| | Levansucrase |
| | Sucrose Phosphorylase |
| | Glycogen Synthase |
| | UDP-Glucuronyltransferase |
| | Galactosyl Transferase |
| | Nucleoside Phosphorylase |
| | a- and B-Amylase |
| | Amyloglucosidase (Glucoamylase) |
| | Cellulase |
| | Dextranase |
| | Chitinase |
| | Pectinase |
| | Lysozyme |
| | Neuraminidase |
| | Xylanase |
| | a- and B-Glucosidase |
| | a- and B-Galactosidase |
| | α- and β-Mannosidase |
| | Invertase |
| | Trahalase |
| | B-N-Acetylglucosaminidase |
| | B-Glucuronidase |
| | Hyaluronidase |
| | B-Xylosidase |
| | Hesperidinase |
| | Pullulanase |
| | a-Fucosidase |
| | Agarase |
| | Endoglycosidase F |
| | NADase |
| | Glycopeptidase F |
| | Thioglucosidase |
| | Cosubstrates/Cofactors: All available sugars and their derivatives. |
| | These sugars can be monosaccharides, disaccharides, and |
| | oligosaccharides and their derivatives. |
| 4. | Etherification of primary and secondary alcohols |
| | Reaction Boxes: A11, B11 |
| | Enzyme Classes: 2.1, 3.2 |
| | Representative Enzymes: |
| | Catechol a-Methyltransferase |
| | Aspartate Transcarbamylase |
| | Ornithine Transcarbamylase |
| | S-Adenosylhomocysteine Hydrolase |
| | Cosubstrates/Cofactors: Alcohols or ethers of any chain length. |
| 5. | Acylation of primary and secondary amines. |
| | Reaction Boxes: E3, E4 |
| | Enzyme Classes: 2.3; 3.1, 3.4, 3.5, 3.6 |
| | Representative Enzymes: |
| | Choline Acetyltransferase |
| | Carnitine Acetyltransferase |
| | Phosphotransacetylase |
| | Chloramphenicol Acetyltransferase |
| | Transglutarninase |
| | gamma-Glutamyl Transpeptidase |
| | Esterases |
| | Lipases |
| | Phospholipase A |
| | Acetylesterase |
| | Acetyl Cholinesterase |
| | Butyryl Cholinesterase |
| | Pectinesterase |
| | Cholesterol Esterase |
| | Glyoxylase II |
| | Alkaline Phosphatase |
| | Acid Phosphatase |
| | A Variety of nucleases |
| | Glucose-6-Phosphatase |
| | Fructose 1,6-Diphosphatase |
| | Ribonuclease |
| | Deoxyribonuclease |
| | Sulfatase |
| | Chondro-4-Sulfatase |
| | Chondro-6-Sulfatase |
| | Leucine Aminopeptidase |
| | Carboxypeptidase A |
| | Carboxypeptidase B |
| | Carboxypeptidase Y |
| | Carboxypeptidase W |
| | Prolidase |
| | Cathepsin C |
| | Chymotrypsin |
| | Trypsin |
| | Elastase |
| | Subtilisin |
| | Papain |
| | Pepsin |
| | Ficin |
| | Bromelin |
| | Rennin |
| | Proteinase A |
| | Collagenase |
| | Urokinase |
| | Asparaginase |
| | Glutaminase |
| | Urease |
| | Acylase I |
| | Penicillinase |
| | Cephalosporinase |
| | Creatininase |
| | Guanase |
| | Adenosine Deaminase |
| | Creatinine Deiminase |
| | Inorganic Pyrophosphatase |
| | ATPase |
| | Cosubstrates/Cofactors: See example number 2, above. |
| 6. | Esterification of carboxylic acids. |
| | Reaction Boxes: 17 |
| | Enzyme Classes: 3.1, 3.6 |
| | Representative Enzymes: |
| | Esterases |
| | Lipases |
| | Phospholipase A |
| | Acetylesterase |
| | Acetyl Cholinesterase |
| | Butyryl Cholinesterase |
| | Pectinesterase |
| | Cholesterol Esterase |
| | Glyoxylase II |
| | Alkaline Phosphatase |
| | Acid Phosphatase |
| | A Variety of nucleases |
| | Glucose-6-Phosphatase |
| | Fructose 1,6-Diphosphatase |
| | Ribonuclease |
| | Deoxyribonuclease |
| | Sulfatase |
| | Chondro-4-Sulfatase |
| | Chondro-6-Sulfatase |
| | Inorganic Pyrophosphatase |
| | APTase |
| | Cosubstrates/Cofactors: alcohols of any chain length being alkyl, aryl, |
| | or their structural derivatives. |

TABLE III
Biocatalytic ‘Reaction Box’ Matrix Approach

| | | A | B | C | D | E | F | G | H | I | J |
|---|---|---|---|---|---|---|---|---|---|---|---|
| Reaction Type | | 1-OH | 2-OH | R₂C═O | R—CHO | 1-NH₂ | 2-NHR | 3-NR₂ | SH(orR) | COOH | COOR |
| Oxidation | 1 | 1 | 1 | | 1 | | | | | | |
| Reduction | 2 | | | 1 | 1 | | | | | | 2 |
| Acylation-Primary | 3 | >30 | | | | >30 | | | >30 | | |
| Acylation-Secondary | 4 | | >30 | | | >30 | | | >30 | | |
| Transesterification | 5 | | | | | | | | | | >30 |
| Interesterification | 6 | | | | | | >30 | >30 | | | >30 |
| Esterification | 7 | | | | | | | | | >100 | |
| Hydrolysis | 8 | | | | | | | | | | |
| Peptide Formation | 9 | | | | | | | | | >100 | |
| Transglycosylation | 10 | >30 | >30 | | | >30 | >30 | >30 | >30 | | |
| Etherification | 11 | >30 | >30 | | | | | | | | |
| Realkylation | 12 | | | | | | 1 | 2 | 1 | | |
| Hydroxylation | 13 | | | | | | | | | | |
| Destination | 14 | | | | | 1 | | | | | |
| Ring Cleavage | 15 | | | | | | | | | | |
| Isomerization | 16 | | | | | | | | | | |
| Ligation | 17 | | | | | | | | | >100 | |
| Oxidative Polymerization | 18 | | | | | | | | | | |
| Hydration/Amination | 19 | | | | | | | | | | |
| Decarboxylation | 20 | | | | | | | | | 1 | |
| Transaid/Ketolases | 21 | | | >30 | >30 | | | | | | |
| Dehalogenation | 22 | | | | | | | | | | |

| | | K | L | M | N | O | P | Q | R | S |
|---|---|---|---|---|---|---|---|---|---|---|
| Reaction Type | | Carbohy | SO₄ | PO₄ | C—X | Ph—NR₂ | Ph—OH | C₆H₅ | C₄C | CONR |
| Oxidation | 1 | 24 | | | | | | | | |
| Reduction | 2 | 1 | 3 | | | | | | | |
| Acylation-Primary | 3 | >30 | | | | | | | | |
| Acylation-Secondary | 4 | >30 × 4 | | | | >30 | >30 | | | |
| Transesterification | 5 | | >30 | >30 | | | | | | |
| Interesterification | 6 | | | | | | | | | >30 |
| Esterification | 7 | | >100 | >100 | | | | | | |
| Hydrolysis | 8 | | 1 | 1 | | | | | | |
| Peptide Formation | 9 | | | | | | | | | |
| Transglycosylation | 10 | >30 | >30 | | | | | | | |
| Etherification | 11 | >30 | | | | | | | | |
| Realkylation | 12 | | | | | 2 | | | | |
| Hydroxylation | 13 | | | | | | >3 | 5 | | |
| Destination | 14 | | | | | 1 | | | | |
| Ring Cleavage | 15 | | | | | | 3 | 4 | | |
| Isomerization | 16 | 1 | | | | | | | | |
| Ligation | 17 | | | | | | | | | |
| Oxidative Polymerization | 18 | | | | | 3 | 3 | | | |
| Hydration/Amination | 19 | | | | | | | | 2 | |
| Decarboxylation | 20 | | | | | | | | | |
| Transaid/Ketolases | 21 | | | | | | | | | |
| Dehalogenation | 22 | | | | 1 | | | | | |

TABLE IV
Reaction Box Analysis of AZT Derivatization
Indicating the Total Number of Possible Reaction Products

| Reaction Box | Number of Possible Products |
|---|---|
| A3 | 30(a) |
| A10 | 30(b) |
| A11 | 30 |
| C2 | 2 × 30^{c} |
| C2 | 2 × 30^{c} |
| G6 | 30 |
| G10 | 30 |
| G12 | ×2 |
| Total | 1-75 × 10¹¹ distinct compounds(d) |

(a)Assuming 30 different acyl donors to be added to this reaction mixture. This includes alkyl, aryl, and of different lengths, (b)Assuming 30 UDP-sugars used in this reaction box, ^{c}Reduction of the hetoner to secondary alcohols leads to the potential acylation of the secondary alcohols and adds 30-fold more potential products; and (d)Each box's possible permutations are multiplied together to estimate the total number of compounds synthesized.

TABLE V
Reaction Box Analysis of Established Drugs
Indicating the Total Number of Possible Reaction Products

| Starting | | Estimated |
|---|---|---|
| Compound | Number of Functional Groups | Number of Derivatives |
| Castanospermine | 4 | 810,000 |
| Cyclosporin | 24 | billions |
| Gentamicin | 8 | billions |
| Haloperidol | 3 | 120 |
| Methotrexate | 7 | greater thatn 10¹⁹ |
| Muscarine | 2 | 2,400 |
| Prazosin | 6 | 288,000 |
| Predniso | | |

| | |
|---|---|
| 1. | KB (Eagle) cell culture assay |
| 2. | Inhibition of the growth of human breast cancer cell lines in vitro |
| 3. | Inhibition of the growth of P388 leukemia cells in vitro |
| 4. | Inhibition of the growth of murine L1210 cells in vitro |
| 5. | Inhibition of gylcinamide ribonucleotide formyltransferase activity |
| 6. | Inhibition of ribonucleotide reductase acitivity |
| 7. | Inhibition of protein kinase C activity |
| 8. | Inhibition of human aromatase activity |
| 9. | Inhibition of DNA topoisomerase II activity |
| 10. | Inhibition of dihydrofolate reductase |
| 11. | Inhibition of aminoimidazole carboxamide ribonucleotide |
| | formyltransferase |

Anti-AIDS Drugs:

| | |
|---|---|
| 1. | Inhibition of HIV virus replication devoid of cytotoxic activity |
| 2 | Inhibition of HIV protease activity |
| 3. | Soluble-formazan assay for HIV-1 |
| 4. | Inhibition of HIV reverse transcriptase activity |

Anti-Hypertensive Drugs:

| | |
|---|---|
| 1. | Inhibition of ACE activity |
| 2. | Inhibition of human plasma renin |
| 3. | Inhibition of in vitro human renin |
| 4. | Inhibition of angiotensin converting enzyme |
| 5. | Alpha 1-adrenergic receptor binding assay |
| 6. | Alpha 2-adrenergic receptor binding assay |
| 7. | Beta-adrenergic receptor binding assay (bronchodilator, cardiotonic, |
| | tocolytic, anti-anginal, anti-arrhythmic, anti-glaucoma) |
| 8. | Dopamine receptor binding assay (anti-migraine, anti-parkinsonian, |
| | anti-emetic, anti-psychotic) |

TABLE VI
Screening Assays to Test for Anti-Cancer, Anti-Viral and
Anti-Hypertensive Activities
Anti-Cancer Drugs

```
  1. KB (Eagle) cell culture assay
  2. Inhibition of the growth of human breast cancer cell lines in vitro
  3. Inhibition of the growth of P388 leukemia cells in vitro
  4. Inhibition of the growth of murine L1210 cells in vitro
  5. Inhibition of gylcinamide ribonucleotide formyltransferase activity
  6. Inhibition of ribonucleotide reductase acitivity
  7. Inhibition of protein kinase C activity
  8. Inhibition of human aromatase activity
  9. Inhibition of DNA topoisomerase II activity
10. Inhibition of dihydrofolate reductase
11. Inhibition of aminoimidazole carboxamide ribonucleotide
    formyltransferase
```

Anti-AIDS Drugs:

1. Inhibition of HIV virus replication devoid of cytotoxic activity
2. Inhibition of HIV protease activity
3. Soluble-formazan assay for HIV-1
4. Inhibition of HIV reverse transcriptase activity

Anti-Hypertensive Drugs:

```
1. Inhibition of ACE activity
2. Inhibition of human plasma renin
3. Inhibition of in vitro human renin
4. Inhibition of angiotensin converting enzyme
5. Alpha 1-adrenergic receptor binding assay
6. Alpha 2-adrenergic receptor binding assay
7. Beta-adrenergic receptor binding assay (bronchodilator, cardiotonic,
    tocolytic, anti-anginal, anti-arrhythmic, anti-glaucoma)
8. Dopamine receptor binding assay (anti-migraine, anti-parkinsonian,
    anti-emetic, anti-psychotic)
```

What is claimed is:

1. A method for drug identification comprising:
(a) conducting a series of biocatalytic reactions by mixing biocatalysts with a starting compound to produce a reaction mixture and thereafter a library of modified starting compounds;
(b) testing the library of modified starting compounds to determine if a modified starting compound is present within the library which exhibits a desired activity;
(c) identifying the specific biocatalytic reactions which produce the modified starting compound of desired activity by systematically eliminating each of the biocatalytic reactions used to produce a portion of the library and testing the compounds produced in the portion of the library for the presence or absence of the modified starting compound with the desired activity; and
(d) repeating the specific biocatalytic reactions which produce the modified compound of desired activity and determining the chemical composition of the reaction product.

2. A method for drug identification of claim 1 wherein
(a) the biocatalytic reactions are conducted with a group of biocatalysts that react with distinct structural moieties found within the structure of a starting compound,
(b) each biocatalyst is specific for one structural moiety or a group of related structural moieties; and
(c) each biocatalyst reacts with many different starting compounds which contain the distinct structural moiety.

3. A method for drug identification comprising:
(a) conducting a series of biocatalytic reactions on a starting compound to produce a library of modified starting compounds;
(b) providing a high affinity receptor for a starting compound of desired activity and exposing the modified compounds formed after each biocatalytic reaction to the high affinity receptor;
(c) isolating the high affinity receptor-modified starting compound complex from the biocatalytic reaction mixture;
(d) dissociating the complex into its component parts; and
(e) determining the chemical composition of the dissociated modified starting compound.

4. A method for drug identification comprising:
(a) adding a high affinity receptor to a starting compound to form a biocatalytic reaction mixture at a concentration that allows for essentially all of the high affinity receptor to bind with the starting compound and about an equal molar amount of starting compound remaining free in solution and available for biocatalytic modification;
(b) producing a modified starting compound exhibiting a higher binding affinity than the starting compound resulting in the displacement of the starting compound from the high affinity receptor;
(c) forming a complex of the modified starting compound and the high affinity receptor, thereby protecting the modified starting compound from further biocatalytic reaction;
(d) isolating the complex from the biocatalytic reaction mixture;
(e) dissociating the complex into its component parts;
(f) determining the chemical composition of the dissociated modified starting compound; and
(g) repeating steps (a) through (b) to produce a library of modified starting compounds.

5. A method for drug identification according to claim 4 wherein the high affinity receptor is present on the surface or contained within a living cell or immobilized to a natural or artificial support.

6. A method for drug identification according to claim 1 wherein the biocatalytic reactions are selected from a group consisting of:
(a) Oxidation of primary and secondary alcohols;
(b) Reduction of aldehydes and ketones;
(c) Acylation of primary and secondary alcohols;
(d) Transglycosylation of primary and secondary alcohols;
(e) Etherification of primary and secondary alcohols;
(f) Acylation of primary and secondary amines; and
(g) Esterification of carboxylic acids.

7. A method for drug identification according to claim 1 wherein the biocatalyst comprises crude or purified enzymes, cellular lysate preparations, partially purified lysate preparations, living cells and intact non-living cells, used in a soluble, suspended or immobilized form.

8. A method for drug identification according to claim 1 wherein the biocatalytic reactions are used in combination with non-specific chemical reactions to produce a library of modified starting compounds.

9. A method for drug identification according to claim 1 further comprising:
(a) exposing the reaction mixture to a drug screening device that measures the binding of compounds with desired activity to localized or immobilized receptor molecules or cells;
(b) correlating a positive measurement from the drug screening device with the sequence of biocatalytic reactions used to synthesize the reaction mixture and the specific reaction sequence producing the modified starting compound with desired activity; and
(c) repeating the biocatalytic reaction sequence to produce the modified starting compound of desired activity and to determine its chemical composition.

10. A method for drug identification according to claim 1 wherein the biocatalytic reaction is performed with a biocatalyst immobilized to magnetic particles forming a magnetic biocatalyst, the method further comprising
(a) initiating the biocatalytic reaction by combining the immobilized biocatalyst with substrate(s), cofactor(s) and solvent/buffer conditions used for a specific biocatalytic reaction;
(b) removing the magnetic biocatalyst from the biocatalytic reaction mixture to terminate the biocatalytic reaction, which is accomplished by applying an external magnetic field causing the magnetic particles with the immobilized biocatalyst to be attracted to and concentrate at the source of the magnetic field, thus effectively separating the magnetic biocatalyst from the bulk of the biocatalytic reaction mixture and allowing for the transferral of the reaction mixture minus the magnetic biocatalyst from a first reaction vessel to a second reaction vessel, leaving the magnetic biocatalyst in the first reaction vessel;
(c) conducting a second biocatalytic reaction, completely independent of the first biocatalytic reaction, by combining a second biocatalyst immobilized to magnetic particles with the second reaction vessel containing the biocatalytic reaction mixture transferred from the first reaction vessel; and
(d) repeating steps a) through c) to accomplish a sequential series of distinct and independent biocatalytic reactions and produce a corresponding series of modified starting compounds.

11. A method for drug identification according to claim 10 wherein the biocatalytic reaction is performed with a biocatalyst immobilized to a particle and the biocatalyst is removed from the biocatalytic reaction mixture by centrifugation or filtration.

12. A method for drug identification according to claim 1 using an automated robotic device, the automated robotic device comprised of:
(a) an XY table with an attached XYZ pipetting boom to add volumetric amounts of enzyme, substrate, cofactor, solvent solutions and assay reagents from reagent vessels positioned on the XY table to reaction and assay vessels positioned on the same XY table;
(b) an XYZ reaction-vessel transfer boom attached to the same XY table used to transfer reaction and assay vessels positioned on the XY table to different locations on the XY table;
(c) a temperature incubation block attached to the same XY table to house the reaction and assay vessels during reaction incubations and control the temperature of the reaction mixtures;
(d) a magnetic separation block attached to the same XY table to separate the biocatalyst immobilized to magnetic particles from the biocatalytic mixture by applying an external magnetic field causing the magnetic particles to be attracted to and concentrate at the source of the magnetic filed, thus effectively separating them from the bulk of the biocatalytic reaction mixture; and
(e) a programmable microprocessor interfaced to the XYZ pipetting boom, and XYZ reaction vessel transfer boom, the temperature block and the magnetic separation block to precisely control and regulate all movements and operations of these functional units in performing biocatalytic reactions to produce modified starting compounds and assays to determined desired activities.

13. A method for rapid drug development comprising:
(a) mixing a starting drug compound with a high affinity receptor at approximately one half the molar concentration of the starting drug compound to allow essentially all of the high affinity receptor to bind to the starting drug compound and leave an equal molar amount of starting drug compound free in solution and available for biocatalytic modification;
(b) producing a modified drug compound from the biocatalytic reaction between the starting drug compound and the receptor, the modified drug compound having a higher binding affinity than the starting drug compound;
(c) displacing the starting drug compound from the receptor with the modified drug compound;
(d) protecting the bound modified drug compound from further biocatalytic reactions;
(e) dissociating the complex of the modified drug compound and receptor into its component parts;
(f) determining the chemical composition of the dissociated modified starting drug compound; and
(g) repeating steps a) through e) and thus conduct a series of biocatalytic reactions on a starting drug compound to produce a library of modified drug compounds with higher binding affinities.

14. A method for rapid drug development according to claim 1 wherein the high affinity receptor is present on the surface or contained within a living cell or immobilized to a natural or artificial support.

15. A method for rapid drug development according to claim 1 wherein the biocatalytic reactions are selected from a group consisting of those listed in Table II.

16. A method for rapid drug development according to claim 1 wherein the testing of the library of modified starting compounds is selected from a group of assays consisting of those listed in Table VI.

17. A method for rapid drug development according to claim 1 wherein the biocatalytic reaction is performed with a biocatalyst immobilized to magnetic particles forming a-magnetic biocatalyst whereas:
(a) the biocatalytic reaction is initiated by combining the immobilized biocatalyst with the following to form the biocatalytic reaction mixture: substrate(s), cofactor(s) and solvent/buffer conditions used for the specific biocatalytic reaction;
(b) the biocatalytic reaction is terminated by removing the magnetic biocatalyst from the biocatalytic reaction mixture, which is accomplished by applying an external magnetic field causing the magnetic particles with the immobilized biocatalyst to be attracted to and concentrate at the source of the magnetic field, thus effectively separating the magnetic biocatalyst from the bulk of the biocatalytic reaction mixture and allowing for the removal of the reaction mixture minus the magnetic biocatalyst to a second reaction vessel, leaving the magnetic biocatalyst in the first reaction vessel;
(c) a second biocatalytic reaction, completely independent of the first biocatalytic reaction, is initiated by adding a second biocatalyst immobilized to magnetic particles to the second reaction vessel containing the biocatalytic reaction mixture from the first biocatalytic reaction minus the magnetic biocatalyst used in the first biocatalytic reaction;
(d) the above described biocatalytic reaction cycle is repeated thus accomplishing a series of distinct and independent biocatalytic reactions producing a corresponding series of modified drug compounds.
(e) a magnetic separation block attached to the same XY table as above to separate the biocatalyst immobilized to magnetic particles from the biocatalytic reaction mixture by applying an external magnetic field causing the magnetic particles to be attracted to and concentrate at the source of the magnetic field, thus effectively separating them from the bulk of the biocatalytic reaction mixture; and
(f) a programmable microprocessor interfaced to the XYZ pipetting boom, the XYZ reaction vessel transfer boom, the temperature block and the magnetic separation block to precisely control and regulate all movements and operations of these functional units in performing biocatalytic reactions to produce modified starting compounds and assays to determined desired activities.
